(19) United States
(12) Patent Application Publication
(10) Pub. No.: US 2007/0224258 A1
(43) Pub. Date: Sep. 27, 2007

(54) Dosage forms having a randomized coating

(76) Inventors: Frank J. Bunick, Randolph, NJ (US); Jen-Chi Chen, Morrisville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/386,606

(22) Filed: Mar. 22, 2006

(51) Int. Cl. A61K 9/48; A61K 9/64

(52) U.S. Cl. 424/451; 424/456

(57) ABSTRACT

A pharmaceutical dosage form is provided comprising a core and a shell adherent thereto, wherein the shell is comprised of at least a first portion and a second portion, each of which is compositionally distinct from each other and arranged in a randomized pattern, such as swirled or marbled. In one embodiment, the shell is comprised of a low temperature, water dispersible film-forming polymer, and first portion and the second portion are visually distinct from each other.

### BACKGROUND OF THE INVENTION

1. Field of the Invention

This invention relates to dosage forms having a randomized coating and a process for their manufacture. More particularly, this invention relates to pharmaceutical dosage forms having a core that is substantially covered with a coating containing a randomized pattern such as marbled or swirled.

2. Background Information

One concern in designing pharmaceutical dosage forms is to ensure that they can be readily identifiable by appearance, e.g., shape, size, markings, color, and surface texture. By including such a functional attribute in dosage form design, subsequent dispensing errors of the medication may be minimized. In addition, patients who take multiple medications can further benefit from such a feature because they can more readily distinguish between the drugs they take at different frequencies and/or times.

One approach for distinguishing dosage forms is to use outer coatings having different colors. However, the range of readily distinguishable colors is somewhat limited. Another approach is to create dosage forms having one color on one end or face, and another color on the other end or face. Although this approach has partially alleviated the problem, there still remains a need for dosage forms having readily identifiable surface appearances.

It is one object of this invention to provide a dosage form having a distinct, randomized surface appearance. It is another object of this invention to provide a dosage form having an outer appearance that is readily distinguishable from other medications. Other objects, features and advantages of the invention will be apparent to those skilled in the art from the detailed description set forth below.

### SUMMARY OF THE INVENTION

The present invention is comprised of, consists of, and/or consists essentially of a dosage form having a core and a coating substantially covering the core, wherein the coating contains a randomized pattern, as well as a method for manufacturing the dosage form, as described in the claims.

### BRIEF DESCRIPTION OF THE DRAWINGS

FIG. 1 is a perspective view of one embodiment of a coated dosage form of the present invention, which has a swirled, randomized surface appearance on its upper face.

FIG. 2 is an enlarged, side cross-sectional view of the dosage form of FIG. 1.

FIG. 3 is a schematic drawing showing an apparatus having a single reservoir and an internal diffusion device therein for use in the process of the present invention.

FIG. 3A is an enlarged, side cross-sectional view of the internal diffusion device used in the apparatus of FIG. 3.

FIG. 3B is an enlarged, perspective view of the internal diffusion device used in the apparatus of FIG. 3.

FIG. 4 is a schematic drawing showing an apparatus having a bi-compartment, single reservoir for use in the process of the present invention.

FIG. 5 is a schematic drawing showing an apparatus having two, independent reservoirs for use in the process of the present invention.

FIG. 6 is a perspective view of one embodiment of a coated dosage form of the present invention, which has a marbilized, randomized surface appearance.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. As used herein, all percentages are by weight unless otherwise specified. In addition, all ranges set forth herein are meant to include any combinations of values between the two endpoints, inclusively.

“Moisture uptake value,” as used herein, shall mean the amount of moisture absorbed by the dosage form when exposed to a temperature of about 40° C. and 75% relative humidity for about 60 minutes, as expressed in terms of percentage relative to the original dosage form weight.

As used herein, the term “dosage form” applies to any ingestible forms, including confections. In one embodiment, dosage forms are solid, semi-solid, or liquid compositions designed to contain a specific pre-determined amount (i.e., e.g., dose) of a certain ingredient, for example an active ingredient as defined below. Suitable dosage forms may be pharmaceutical drug delivery systems, including those for oral administration, buccal administration, rectal administration, topical, transdermal, or mucosal delivery, or subcutaneous implants, or other implanted drug delivery systems; or compositions for delivering minerals, vitamins and other nutraceuticals, oral care agents, flavorants, and the like. In one embodiment, the dosage forms of the present invention are considered to be solid; however, they may contain liquid or semi-solid components. In another embodiment, the dosage form is an orally administered system for delivering a pharmaceutical active ingredient to the gastrointestinal tract of a human. In yet another embodiment, the dosage form is an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

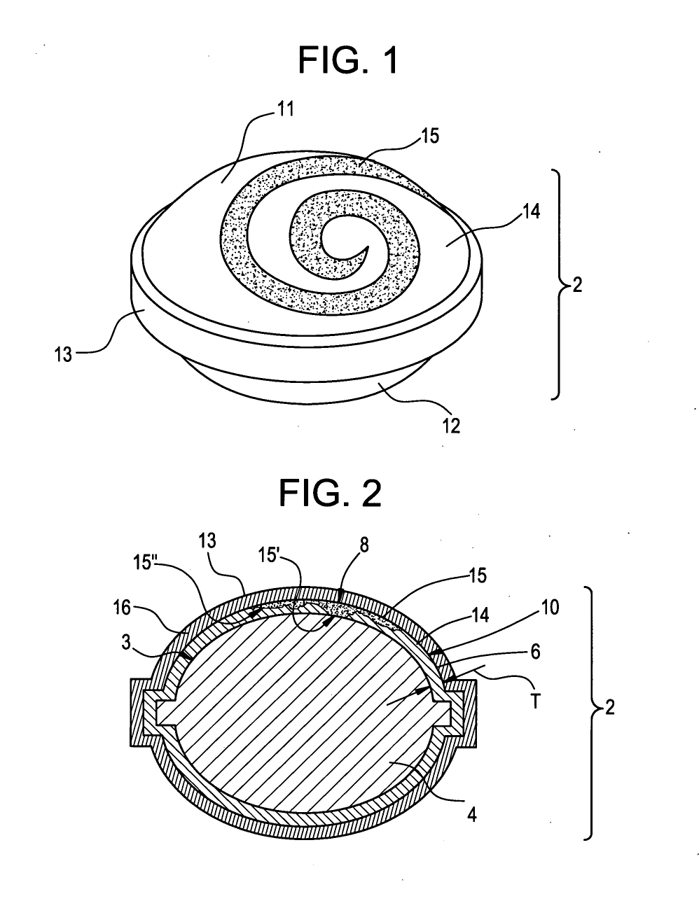

“Tablets,” as used herein, refer to compressed or molded solid dosage forms of any shape or size. As illustrated in FIG. 1, one type of tablet has an upper face 11 and a lower face 12 opposed thereto, which are formed by the upper and lower punch faces, respectively, as well as a bellyband 13 defined during compaction via contact with a die wall.

“Low temperature thermally responsive material,” as used herein, shall mean a material that forms a film at temperatures less than about 100° C.

By “randomized pattern,” it is meant one that is marbled, veined, helical, swirled, spiraled, twisted, curved, streaked, or striated. As used herein, “marbled” is meant to be a randomized pattern that is streaked and mottled with veins and colors to imitate marble stone, whereby such streaks may be, for example, circular or varied in shape and/or pattern. As used herein, “swirled” is meant a randomized pattern that is twisted in a circular, whirlpool-like manner. In one embodiment, a marbled randomized pattern may include a swirled pattern.

As used herein, “low temperature water dispersible thermally responsive materials” shall mean those thermally responsive materials that form a film at temperatures below 100° C. and are water dispersible. “Water soluble” or “water solubilize,” as used herein in connection with non-polymeric materials, shall mean from sparingly soluble to very soluble, i.e., not more than 100 parts water required to dissolve 1 part of the non-polymeric, water soluble solute. See Remington, “The Science and Practice of Pharmacy,” pages 208-209 (2000). “Water soluble” or “water solubilize,” as used herein in connection with polymeric materials, shall mean that the polymer swells in water and can be dispersed at the molecular level to form a homogeneous dispersion or colloidal solution. “Water dispersible,” as used herein in connection with polymeric materials, shall mean at least a portion of the polymer is removed from the dosage form within 60 minutes after immersion of the dosage form in an aqueous medium such as that used for in-vitro dissolution testing, or gastrointestinal fluids.

As used herein, “injection molding” shall mean a process of forming a dosage form in a desired shape and size wherein a flowable material, which is in a fluid or flowable state form, enters a mold, then is solidified in the mold via a change in temperature (either positive or negative) before being removed therefrom. By contrast, “compression,” as used herein, shall mean a process of forming a dosage form in a desired shape and size wherein a material is compacted into a tablet between the surfaces of punches via an increase in pressure before being removed therefrom.

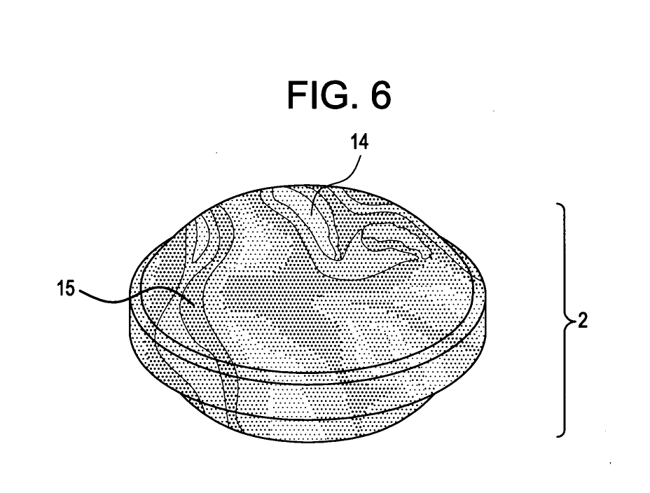

As used herein, the term “compositionally different” means having features that are readily distinguishable by qualitative or quantitative chemical analysis, physical testing, or visual observation. For example, as illustrated in FIGS. 1 and 2, the first portion 14 and second portion 15 of the shell 6 may contain different ingredients, or different levels of the same ingredients, or the first and second materials may have different physical or chemical properties, different functional properties, and/or be visually distinct. As illustrated in FIG. 6, first portion 14 and second portion 15 can be in a veined, randomized pattern, and substantially all or portions of such pattern may extend through the thickness of the coating. Examples of physical or chemical properties that may be different include hydrophylicity, hydrophobicity, hygroscopicity, elasticity, thickness, porosity, plasticity, tensile strength, crystallinity, and density. Examples of functional properties which may be different include rate and/or extent of dissolution of the material itself or of an active ingredient therefrom, rate of disintegration of the material, permeability to active ingredients, permeability to water or aqueous media, and the like. Examples of visual distinctions include size, shape, topography, or other geometric features, color, hue, opacity, reflective qualities, brightness, depth, shades, chroma, gloss, and the like.

For example, the shell could have at least two portions having different visual appearances as follows: a white portion and a blue portion (such as a white background having a blue swirl thereon), or a flat finish portion and a glossy portion, or an opaque portion and a translucent portion. While the apparatus and methods of the present invention will be discussed hereinafter as employing shells that have differently colored swirls (i.e., white and blue swirls) it will be understood that the patterned films may have any of the foregoing types of compositionally different portions, or combinations thereof, including, but not limited to, visual distinctions not specifically mentioned herein.

As depicted in the cross-sectional side view of FIG. 2, one embodiment of the present invention is directed to a dosage form 2 comprised of a core 4 having an outer core surface 3 that is substantially covered by a coating or shell 6. More particularly, as will be described in further detail hereinafter, the core 4 of the present invention is covered by a coating 6 having a randomized pattern with at least two compositionally distinct portions, i.e., e.g., at least two portions having different visual appearances. It is noted that, hereinafter, the apparatus and method of the present invention are discussed as producing dosage forms comprised of cores that are substantially coated by the film or films and the term “substantially” shall be understood to mean that at least about 95% of the surface area of the core, or at least about 95% of at least one face, is covered by the film or films. Furthermore, it will be understood by those having ordinary skill in the art that the apparatus and method of the present invention may also be adapted to produce coated dosage form products that are at least partially covered by the film or films. The term “at least partially covered” shall be understood to mean that at least about 25% to about 100% of the surface area of the core is covered by the film or films.

In one embodiment, the dried coating or shell 6 is comprised of at least a first portion 14 having a first feature and a second portion 15 having a second feature, and the first and second features are compositionally different from each other and arranged in a randomized pattern. For example, as illustrated in FIG. 1, the shell 6 is comprised of at least a first portion 14 having a first visual appearance and a second portion 15 having a second visual appearance, and the first and second portions are visually distinct from each other and arranged in a randomized, swirled pattern.

FIG. 2 is a cross-sectional view of the dosage form 2 of FIG. 1. As shown, the coating or shell 6 has a first portion 14 and a second portion 15, whereby the second portion forms a randomized pattern in the coating, and has a thickness that varies across the outer core surface 3 of the core 4. Depending upon, for example, the randomized pattern selected and the extent of blending between the flowable material of the first portion 14 and the flowable material of the second portion 15, the thickness of second portion 15 may extend partially into the first portion 14 from the first upper or top surface 8 or may extend through the first portion 14 and contact the outer core surface 3 of the core 4. For example, at some locations 15′, the second portion may have a thickness that extends to or proximate to the outer core surface 3, whereas at other locations 15″, the second portion may have a thickness that does not fully extend to or is proximate to the outer core surface 3. In another embodiment (not shown), the second portion 15 may have a thickness that is substantially consistent across the outer core surface 3, i.e., e.g. either the thickness extends to or is proximate to the outer core surface 3 or the thickness is substantially consistent at some point between the outer core surface 3 and the upper first surface 8 of the shell 6.

In another embodiment as illustrated in FIG. 6, the second portion 15 may be present in multiple faces of the dosage form, including the upper, lower faces and/or the bellyband of the dosage form.

Although FIG. 2 depicts the upper surface 8 of the second portion 15 as being substantially uniform with the proximate upper surface 10 of the first portion 14, the upper surface 8 of the second portion 15 alternative may protrude from or be recessed from the proximate upper surface 10 of the first portion 14, either at locations or substantially consistently across the upper shell surface 8.

The thickness, shown as “T” in FIG. 2, of the shell 6 may vary depending upon, for example, the surface area of core to be coated, the desired shell appearance, and/or the desired shell composition, but will generally range from about 10 microns to about 5000 microns. The shell also generally covers a surface area of from about greater than 0% to less than about 100% of a dosage form face, e.g., greater than about 10% and less than about 90% or greater than about 25% and less than about 50%. “Face,” as used herein, is the portion of a compressed tablet formed by the upper and lower punch faces, and includes one-half of the overlap area of a rim as illustrated in United States Patent Application Publication No. 20040109889.

In one embodiment, the dosage form contains one or more active ingredients. “Active ingredients,” as used herein, includes, for example, pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, diuretics, expectorants, gastrointestinal agents, migraine preparations, motion sickness products, mucolytics, muscle relaxants, osteoporosis preparations, polydimethylsiloxanes, respiratory agents, sleep-aids, urinary tract agents and mixtures thereof.

Suitable oral care agents include breath fresheners, tooth whiteners, antimicrobial agents, tooth mineralizers, tooth decay inhibitors, topical anesthetics, mucoprotectants, and the like.

Suitable flavorants include menthol, peppermint, mint flavors, fruit flavors, chocolate, vanilla, bubblegum flavors, coffee flavors, liqueur flavors and combinations and the like.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotadine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate and loperamide; glycopyrrolate; antiemetics, such as ondansetron, analgesics, such as mesalamine.

In one embodiment of the invention, the active ingredient may be selected from bisacodyl, famotadine, ranitidine, cimetidine, prucalopride, diphenoxylate, loperamide, lactase, mesalamine, bismuth, antacids, and pharmaceutically acceptable salts, esters, isomers, and mixtures thereof.

In another embodiment, the active ingredient may be selected from analgesics, anti-inflammatories, and antipyretics: e.g. non-steroidal anti-inflammatory drugs (NSAIDs), including propionic acid derivatives: e.g. ibuprofen, naproxen, ketoprofen and the like; acetic acid derivatives: e.g. indomethacin, diclofenac, sulindac, tolmetin, and the like; fenamic acid derivatives: e.g. mefanamic acid, meclofenamic acid, flufenamic acid, and the like; biphenylcarbodylic acid derivatives: e.g. diflunisal, flufenisal, and the like; and oxicams, e.g. piroxicam, sudoxicam, isoxicam, meloxicam, and the like. In one embodiment, the active ingredient is selected from propionic acid derivative NSAID: e.g. ibuprofen, naproxen, flurbiprofen, fenbufen, fenoprofen, indoprofen, ketoprofen, fluprofen, pirprofen, carprofen, oxaprozin, pranoprofen, suprofen, and pharmaceutically acceptable salts, derivatives, and combinations thereof. In another embodiment of the invention, the active ingredient may be selected from acetaminophen, acetyl salicylic acid, ibuprofen, naproxen, ketoprofen, flurbiprofen, diclofenac, cyclobenzaprine, meloxicam, rofecoxib, celecoxib, and pharmaceutically acceptable salts, esters, isomers, and mixtures thereof.

In another embodiment of the invention, the active ingredient may be selected from pseudoephedrine, phenylpropanolamine, phenylephrine, chlorpheniramine, dextromethorphan, diphenhydramine, astemizole, terfenadine, fexofenadine, loratadine, desloratidine, doxilamine, norastemizole, cetirizine, guaifenesin, benzocaine, menthol, modafinil, nifedipene, sidenefil, mixtures thereof and pharmaceutically acceptable salts, esters, isomers, and mixtures thereof.

Examples of suitable polydimethylsiloxanes, which include, but are not limited to dimethicone and simethicone, are those disclosed in U.S. Pat. Nos. 4,906,478, 5,275,822, and 6,103,260. As used herein, the term “simethicone” refers to the broader class of polydimethylsiloxanes, including but not limited to simethicone and dimethicone.

The active ingredient or ingredients are present in the dosage forms of the present invention in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors must be considered, as known in the art. In one embodiment, the dosage form comprises at least about 85 weight percent of the active ingredient.

The active ingredient or ingredients may be present in the dosage form in any form. For example, the active ingredient may be dispersed at the molecular level, e.g. melted or dissolved, within the dosage form, or may be in the form of particles, which in turn may be coated or uncoated. Particles may be present in the shell and/or the core of the dosage form. If the active ingredient is in form of particles, the particles (whether coated or uncoated) typically have an average particle size of about 1 micron to about 2000 microns. In one embodiment, such particles are crystals having an average particle size of about 1 micron to about 300 microns. In yet another embodiment, the particles are granules or pellets having an average particle size of about 50 microns to about 2000 microns, e.g. from about 50 microns to about 1000 microns or from about 100 microns to about 800 microns.

In certain embodiments in which modified release of the active ingredient is desired, the active ingredient may optionally be coated with a known release-modifying coating. This advantageously provides an additional tool for modifying the release profile of active ingredient from the dosage form. For example, the dosage form may contain coated particles of one or more active ingredients, in which the particle coating confers a release modifying function, as is well known in the art. Examples of suitable release modifying coatings for particles are described in U.S. Pat. Nos. 4,173,626; 4,863,742; 4,980,170; 4,984,240; 5,286,497; 5,912,013; 6,270,805; and 6,322,819. Commercially available modified release active ingredients may also be employed. For example, acetaminophen particles, which are encapsulated with release-modifying polymers by a coaccervation process, may be used in the present invention. Such coaccervation-encapsulated acetaminophen is commercially available from, for example, Eurand America, Inc. or Circa Inc.

If the active ingredient has an objectionable taste, and the dosage form is intended to be chewed or disintegrated in the mouth prior to swallowing, the active ingredient may be coated with a taste masking coating, as known in the art. Examples of suitable taste masking coatings are described in, for example, U.S. Pat. Nos. 4,851,226; 5,075,114; and 5,489,436. Commercially available taste masked active ingredients may also be employed. For example, acetaminophen particles, which are encapsulated with ethylcellulose or other polymers by a coaccervation process, may be used in the present invention. Such coaccervation-encapsulated acetaminophen is commercially available from Eurand America, Inc. or Circa Inc. Additional suitable methods for applying taste-masked coatings are well known in the art and include but are not limited to fluid bed coating, complex coaccervation, spray drying, and spray congealing as disclosed in, for example, U.S. Pat. Nos. 4,851,226, 5,653,993, 5,013,557, and 6,569,463, respectively.

The active ingredient or ingredients are typically capable of dissolution upon contact with a fluid such as water, stomach acid, intestinal fluid or the like. In one embodiment, the dissolution characteristics of the active ingredient meet USP specifications for immediate release tablets containing the active ingredient. In embodiments in which it is desired for the active ingredient to be absorbed into the systemic circulation of an animal, the active ingredient or ingredients should be capable of dissolution upon contact with a fluid such as water, gastric fluid, intestinal fluid or the like. In one embodiment, the dissolution characteristics of the active ingredient meet USP specifications for immediate release tablets containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing, and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes after dosing. See USP 24, 2000 Version, 19-20 and 856 (1999). In another embodiment, the dissolution characteristics of the active ingredient may be modified: e.g. controlled, sustained, extended, retarded, prolonged, or delayed.

The core may also optionally comprise a sub-core (which may also be referred to as an “insert”), which may be made by any method, for example compression or molding, and which may optionally contain one or more active ingredients.

The core (or substrate) may be any solid or semi-solid form. As used herein, “substrate” refers to a surface or underlying support, upon which another substance resides or acts, and “core” refers to a material, which is at least partially enveloped or surrounded by another material. In one embodiment, the core comprises a solid, for example, the core may be a compressed or molded tablet, hard or soft capsule, suppository, or a confectionery form such as a lozenge, nougat, caramel, fondant, or fat based composition. In certain other embodiments, the core may be in the form of a semi-solid or a liquid in the finished dosage form.

The core of the present invention may be prepared by any suitable method, including for example compression and molding, and depending on the method by which it is made, typically comprises active ingredient and a variety of excipients, i.e., inactive ingredients which may be useful for conferring desired physical properties to the dosage core.

In embodiments wherein the core is a compressed dosage form, for example, a compressed tablet, the core may be obtained from a compressed powder. The powder may contain an active ingredient, and optionally comprise various excipients, such as binders, disintegrants, lubricants, fillers and the like, as is conventional, or the powder may comprise other particulate material of a medicinal or non-medicinal nature, such as inactive placebo blends for tableting, confectionery blends, and the like. One particular formulation comprises active ingredient, as an excipient, a plastically deforming compressible material, and optionally other excipients, such as disintegrants and lubricants and is described in more detail in United States Patent Application Publication No. 20030068373. During compression, the plastically deforming compressible material assumes the shape of the microrelief from the upper and/or lower punch surface.

Suitable plastically deforming compressible materials for these embodiments include: microcrystalline cellulose, waxes, fats, mono- and di-glycerides, derivatives and mixtures thereof, and the like. In certain embodiments, wherein the plastically deforming compressible material is later caused to melt and be absorbed into the tablet, the plastically deforming compressible material may be selected from low-melting plastically deforming compressible materials, such as plastically deforming compressible powdered waxes, such as shellac wax and microcrystalline wax, polyethylene glycol, and mixtures thereof.

Suitable fillers include, but are not limited to, water-soluble compressible carbohydrates such as sugars, which include dextrose, sucrose, isomaltalose, fructose, maltose, and lactose, polydextrose, sugar-alcohols, which include mannitol, sorbitol, isomalt, maltitol, xylitol, erythritol, starch hydrolysates, which include dextrins, and maltodextrins, and the like, water insoluble plastically deforming materials such as microcrystalline cellulose or other cellulosic derivatives, water-insoluble brittle fracture materials such as dicalcium phosphate, tricalcium phosphate and the like and mixtures thereof.

Suitable binders include, but are not limited to, dry binders such as polyvinyl pyrrolidone, hydroxypropylmethylcellulose, and the like; wet binders such as water-soluble polymers, including hydrocolloids such as alginates, agar, guar gum, locust bean, carrageenan, tara, gum arabic, tragacanth, pectin, xanthan, gellan, maltodextrin, galactomannan, pusstulan, pullulan, laminarin, scleroglucan, gum arabic, inulin, pectin, whelan, rhamsan, zooglan, methylan, chitin, cyclodextrin, chitosan, polyvinyl pyrrolidone, cellulosics, starches, and the like; and derivatives and mixtures thereof.

Suitable disintegrants include, but are not limited to, sodium starch glycolate, cross-linked polyvinylpyrrolidone, cross-linked carboxymethylcellulose, starches, microcrystalline cellulose, and the like.

Suitable lubricants include, but are not limited to, long chain fatty acids and their salts, such as magnesium stearate and stearic acid, talc, and waxes.

Suitable glidants include, but are not limited to, colloidal silicon dioxide, and the like.

In embodiments in which the core is prepared via compression, the core may also incorporate pharmaceutically acceptable adjuvants, including, but not limited to preservatives, high intensity sweeteners such as aspartame, acesulfame potassium, cyclamate, saccharin, sucralose, and the like; and other sweeteners such as dihydroalcones, glycyrrhizin, Monellin™, stevioside, Talin™, and the like; flavors, antioxidants, surfactants, and coloring agents.

In one embodiment of the invention, the dosage forms of this invention comprise a core made from a blend of powders having an average particle size of about 50 microns to about 500 microns. In one embodiment, the active ingredient has an average particle size of about 50 microns to about 500 microns. In another embodiment, at least one excipient has an average particle size of about 50 microns to about 500 microns, e.g. about 100 to about 500 microns. In one such embodiment, a major excipient, i.e. an excipient comprising at least 50% by weight of the core, has an average particle size of about 50 microns to about 500 microns, e.g. about 100 to about 500 microns. Particles in this size range are particularly useful for direct compression processes.

In one embodiment of the invention, the core may be a directly compressed tablet made from a powder that is substantially free of water soluble polymeric binders and hydrated polymers. This composition is advantageous for maintaining an immediate release dissolution profile, minimizing processing and material costs, and providing for optimal physical and chemical stability of the dosage form.

In embodiments in which the core is prepared by direct compression, the materials comprising the core, e.g. the active ingredient or ingredients and excipients, may be blended together, for example as dry powders, and fed into a cavity of an apparatus that applies pressure to form a core. Any suitable compacting apparatus may be used, including for example a roller compactor such as a chilsonator or drop roller; or a conventional tablet press. In one embodiment, the core may be formed by compaction using a rotary tablet press as known in the art. In general, a metered volume of powder is filled into a die cavity of the rotary tablet press, and the cavity rotates as part of a “die table” from the filling position to a compaction position. At the compaction position, the powder is compacted between an upper and a lower punch, then the resulting tablet is pushed from the die cavity by the lower punch. Advantageously, the direct compression process enables the minimization or elimination of water-soluble, non-saccharide polymeric binders such as polyvinyl pyrrolidone, alginates, hydroxypropyl cellulose, hydroxypropylmethylcellulose, hydroxyethylcellulose, and the like, which could have a negative effect on dissolution.

In another embodiment, the core may be prepared by the compression methods and apparatus described in United States Patent Application Publication No. 20040156902. Specifically, the core may be made using a rotary compression module comprising a fill zone, insertion zone, compression zone, ejection zone, and purge zone in a single apparatus having a double row die construction as shown in FIG. 6 of United States Patent Application Publication No. 20040156902. The dies of the compression module may then be filled using the assistance of a vacuum, with filters located in or near each die. The purge zone of the compression module includes an optional powder recovery system to recover excess powder from the filters and return the powder to the dies.

In another embodiment, the core may be prepared by a wet-granulation method, in which the active ingredient or ingredients, appropriate excipients, and a solution or dispersion of a wet binder (e.g. an aqueous cooked starch paste, or solution of polyvinyl pyrrolidone) may be mixed and granulated. Suitable apparatus for wet granulation include low shear, e.g. planetary mixers, high shear mixers, and fluid beds, including rotary fluid beds. The resulting granulated material may then be dried, and optionally dry-blended with further ingredients, e.g. adjuvants and/or excipients such as, for example, lubricants, colorants, and the like. The final dry blend is then suitable for compression by the methods described in the previous paragraph.

Methods for direct compression and wet granulation processes are known in the art, and are described in detail in, for example, Lachman, et al., The Theory and Practice of Industrial Pharmacy, Chapter 11 (3rd ed. 1986).

In one embodiment, the shell or core may also be prepared by thermal setting injection molding using the method and apparatus in which the mold is maintained at approximately a constant temperature as described in United States Patent Application Publication No. 20030124183. In this embodiment, the first portion or core may be formed by injecting a starting material in flowable form into a molding chamber. The starting material may comprise an active ingredient and a thermally responsive material, which is introduced to the mold at a temperature above the glass transition temperature or set temperature of the thermally responsive material but below the decomposition temperature of the active ingredient. The starting material is then cooled and solidified in the molding chamber into a desired shaped form (i.e. the shape of the mold). The starting material, when at a temperature that is greater than its glass transition temperature or its set temperature, is sufficiently flowable to be easily injected or pumped into the molding chamber.

As used herein, “thermally responsive material” shall include materials that, as the temperature applied to the material is increased, become softer, and as the temperature applied is reduced, the materials conversely becomes harder and have reduced flow. In the case of gels, “set temperature” shall mean the temperature at which a gel-forming material rapidly solidifies through the gelation process.

In another embodiment, the shell or core may be prepared by thermal cycle injection molding using the method and apparatus, in which the mold is cycled between at least two temperatures, as described in United States Patent Application Publication No. 20030086973. In this embodiment, the first portion or core may be formed by injecting a starting material in flowable form into a heated molding chamber. The starting material may comprise an active ingredient and a thermoplastic material at a temperature above the glass transition temperature or set temperature of the thermally responsive material but below the decomposition temperature of the active ingredient. The starting material is then cooled and solidified in the molding chamber into a desired shaped form (i.e. the shape of the mold).

According to either of these molding methods, the starting material must be in flowable form. For example, it may comprise solid particles suspended in a molten matrix such as a polymer matrix. Alternatively, the starting material may be completely molten or in the form of a paste. In one embodiment, the starting material may comprise an active ingredient dissolved in a molten material. Alternatively, the starting material may be made by dissolving a solid in a solvent, which solvent may then be evaporated from the starting material after it has been molded.

The starting material may comprise any edible material which is desirable to incorporate into a shaped form, including active ingredients such as those active ingredients previously described with respect to the core, nutritionals, vitamins, minerals, flavors, sweeteners, and the like. Typically, the starting material comprises an active ingredient and a thermally responsive material. The thermally responsive material may be any edible material that is flowable at a temperature between about 37° C. and about 250° C., and that is a solid or semi-solid at a temperature between about −10° C. and about 35° C. When it is in the fluid or flowable state, the flowable starting material may comprise a dissolved or molten component, and optionally a solvent such as for example water or organic solvents, or combinations thereof. The solvent may be partially or substantially removed by drying.

Suitable flowable, starting materials include, but are not limited to those thermally responsive materials such as film forming polymers, gelling polymers, hydrocolloids, low melting hydrophobic materials such as fats and waxes, non-crystallizable carbohydrates, and the like.

Examples of suitable thermally responsive materials include, but are not limited to water-soluble polymers such as polyalkylene glycols, polyethylene oxides and derivatives, and sucrose-fatty acid esters; fats such as cocoa butter, hydrogenated vegetable oil such as palm kernel oil, cottonseed oil, sunflower oil, and soybean oil; free fatty acids and their salts; mono- di- and triglycerides, phospholipids, waxes such as carnuba wax, spermaceti wax, beeswax, candelilla wax, shellac wax, microcrystalline wax, and paraffin wax; fat-containing mixtures such as chocolate; sugar in the form of an amorphous glass such as that used to make hard candy forms, sugar in a supersaturated solution such as that used to make fondant forms; carbohydrates such as sugar-alcohols (for example, sorbitol, maltitol, mannitol, xylitol and erythritol), or thermoplastic starch; and low-moisture polymer solutions such as mixtures of gelatin and other hydrocolloids at water contents up to about 30%, such as for example those used to make “gummi” confection forms. In one embodiment, the thermally responsive material is a blend of fats and mono- and diglycerides.

In one embodiment of the invention, the flowable materials may comprise a film former such as a cellulose ether, e.g. hydroxypropylmethylcellulose or a modified starch, e.g. waxy maize starch; optionally a polycarbohydrate, e.g. maltodextrin; optionally a hydrocolloid, e.g. xanthan gum or carrageenan, or a sugar, e.g. sucrose; and optionally a plasticizer such as polyethylene glycol, propylene glycol, vegetable oils such as castor oil, glycerin, and mixtures thereof.

Any film former known in the art is also suitable for use as a thermally responsive material. Examples of suitable film formers include, but are not limited to, polyvinylalcohol (PVA), polyvinylpyrrolidone (PVP), hydroxypropyl starch, hydroxyethyl starch, pullulan, methylethyl starch, carboxymethyl starch, methylcellulose, hydroxypropylcellulose (HPC), hydroxyethylmethylcellulose (HEMC), hydroxypropylmethylcellulose (HPMC), hydroxybutylmethylcellulose (HBMC), hydroxyethylethylcellulose (HEEC), hydroxyethylhydroxypropylmethyl cellulose (HEMPMC), methacrylic acid and methacrylate ester copolymers, polyethylene oxide and polyvinylpyrrolidone copolymers, gelatin, proteins such as whey protein, coaggulatable proteins such as albumin, casein, and casein isolates, soy protein and soy protein isolates, pre-gelatinized starches, and polymers and derivatives and mixtures thereof.

One suitable hydroxypropylmethylcellulose compound is HPMC 2910, which is a cellulose ether having a degree of substitution of about 1.9 and a hydroxypropyl molar substitution of 0.23, and containing, based upon the total weight of the compound, from about 29% to about 30% methoxyl groups and from about 7% to about 12% hydroxylpropyl groups. HPMC 2910 is commercially available from the Dow Chemical Company under the tradename, “METHOCEL E.” METHOCEL E5, which is one grade of HPMC-2910 suitable for use in the present invention, has a viscosity of about 4 to 6 cps (4 to 6 millipascal-seconds) at 20° C. in a 2% aqueous solution as determined by a Ubbelohde viscometer. Similarly, METHOCEL E6, which is another grade of HPMC-2910 suitable for use in the present invention, has a viscosity of about 5 to 7 cps (5 to 7 millipascal-seconds) at 20° C. in a 2% aqueous solution as determined by a Ubbelohde viscometer. METHOCEL E15, which is another grade of HPMC-2910 suitable for use in the present invention, has a viscosity of about 15000 cps (15 millipascal-seconds) at 20° C. in a 2% aqueous solution as determined by a Ubbelohde viscometer. As used herein, “degree of substitution” shall mean the average number of substituent groups attached to a anhydroglucose ring, and “hydroxypropyl molar substitution” shall mean the number of moles of hydroxypropyl per mole anhydroglucose.

As used herein, “modified starches” include starches that have been modified by crosslinking, chemically modified for improved stability, or physically modified for improved solubility properties. As used herein, “pre-gelatinized starches” or “instantized starches” refers to modified starches that have been pre-wetted, then dried to enhance their cold-water solubility. Suitable modified starches are commercially available from several suppliers such as, for example, A.E. Staley Manufacturing Company, and National Starch & Chemical Company. One suitable modified starch includes the pre-gelatinized waxy maize derivative starches that are commercially available from National Starch & Chemical Company, such as PURITY GUM 59, and derivatives, copolymers, and mixtures thereof. Such waxy maize starches typically contain, based upon the total weight of the starch, from about 0 percent to about 18 percent of amylose and from about 100% to about 88% of amylopectin.

Suitable tapioca dextrins include those available from National Starch & Chemical Company under the tradenames “CRYSTAL GUM” or “K-4484,” and derivatives thereof such as modified food starch derived from tapioca, which is available from National Starch and Chemical under the tradename, “PURITY GUM 40,” and copolymers and mixtures thereof.

Examples of suitable hydrocolloids (also referred to herein as gelling polymers) include but are not limited to alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, pectin, whelan, rhamsan, zooglan, methylan, chitin, chitosan, and derivatives and mixtures thereof.

Suitable xanthan gums include those available from C.P. Kelco Company under the tradenames, “KELTROL 1000,” “XANTROL 180,” or “K9B310.”

Thermoplastic materials that can be molded and shaped when heated are suitable for use as the thermally responsive material, and include both water soluble and water insoluble polymers that are generally linear, not crosslinked, nor strongly hydrogen bonded to adjacent polymer chains. Examples of suitable thermoplastic materials include: chemically modified cellulose derivatives such as hydroxypropyl cellulose (HPC), hydroxypropylmethyl cellulose (HPMC), methyl cellulose (MC), cellulose acetate (CA), ethyl cellulose (EC), cellulose acetate butyrate (CAB), cellulose propionate; vinyl polymers such as polyvinyl alcohol (PVA) and polyvinyl pyrrolidone (PVP); thermoplastic starch; thermoplastic gelatin, natural and chemically modified proteins such as gelatin, soy protein isolates, whey protein, myofibrillar proteins, and the milk derived caseinate proteins; and derivatives and combinations thereof.

Any plasticizer known in the pharmaceutical art is suitable for use in the flowable material, and may include, but not be limited to polyethylene glycol; glycerin; sorbitol; triethyl citrate; tribuyl citrate; dibutyl sebecate; vegetable oils such as castor oil; surfactants such as polysorbates, sodium lauryl sulfates, and dioctyl-sodium sulfosuccinates; propylene glycol; mono acetate of glycerol; diacetate of glycerol; triacetate of glycerol; natural gums and mixtures thereof. In solutions containing a cellulose ether film former, an optional plasticizer may be present in an amount, based upon the total weight of the solution, from about 0% to about 40%.

Any thickener known in the art may optionally be added to the thermally responsive material. Additional suitable thickeners include, but are not limited to, cyclodextrin, crystallizable carbohydrates, and the like, and derivatives and combinations thereof. Suitable crystallizable carbohydrates include the monosaccharides and the oligosaccharides. Of the monosaccharides, the aldohexoses e.g., the D and L isomers of allose, altrose, glucose, mannose, gulose, idose, galactose, talose, and the ketohexoses e.g., the D and L isomers of fructose and sorbose along with their hydrogenated analogs: e.g., glucitol (sorbitol), and mannitol are preferred. Of the oligosaccharides, the 1,2-disaccharides sucrose and trehalose, the 1,4-disaccharides maltose, lactose, and cellobiose, and the 1,6-disaccharides gentiobiose and melibiose, as well as the trisaccharide raffinose are preferred along with the isomerized form of sucrose known as isomaltulose and its hydrogenated analog isomalt. Other hydrogenated forms of reducing disaccharides (such as maltose and lactose), for example, maltitol and lactitol are also preferred. Additionally, the hydrogenated forms of the aldopentoses: e.g., D and L ribose, arabinose, xylose, and lyxose and the hydrogenated forms of the aldotetroses: e.g., D and L erythrose and threose are suitable and are exemplified by xylitol and erythritol, respectively.

The flowable material may optionally comprise adjuvants or excipients, which may comprise up to about 20% by weight of the flowable material. Examples of suitable adjuvants or excipients include detackifiers, humectants, surfactants, anti-foaming agents, colorants, flavorants, sweeteners, opacifiers, and the like. In one embodiment, the flowable material comprises less than 5% humectants, or alternately is substantially free of humectants, such as glycerin, sorbitol, maltitol, xylitol, or propylene glycol. Humectants have traditionally been included in pre-formed films employed in enrobing processes, such as that disclosed in U.S. Pat. Nos. 5,146,730 and 5,459,983 to ensure adequate flexibility or plasticity and bondability of the film during processing. Humectants function by binding water and retaining it in the film. Pre-formed films used in enrobing processes can typically comprise up to 45% water. Disadvantageously, the presence of humectant prolongs the drying process, and can adversely affect the stability of the finished dosage form.

In another embodiment, the core may be a hollow or evacuated core. For example, the core may be an empty capsule shell. Alternatively, a hollow core may be prepared for example by injection molding or shell molding. In one such method, flowable material is injected into a mold cavity, then cavity is brought to a temperature at which the outer surface of the core (which is in contact with the mold) begins to solidify or set. The excess flowable material from the center of the core is then withdrawn from the mold using suitable means, for example a piston pump. In another such method, an empty capsule is used as a sub-core, and a coating layer is formed thereon by methods known in the art such as, for example, spray-coating, dip-coating, injection cycle molding as described in, for example, United States Patent Application Publication No. 20030086973. In certain embodiments of the invention, the core may further comprise any of the aforementioned subcoatings applied by any method known in the art, for example spraying, compression, or molding. In certain other embodiments of the invention, the core may be substantially free of a subcoating.

In another embodiment of the invention, the core contains at least in part one or more inserts. The inserts can be made in any shape or size. For instance, irregularly shaped inserts can be made, that is shapes having no more than one axis of symmetry. Cylindrically shaped inserts may also be made. The insert may be made using conventional techniques such as panning, compression, or molding. In one embodiment, the insert is prepared using the injection molding methods and apparatus as described herein.

In one embodiment of the invention, the insert may have an average diameter from about 100 to about 1000 microns. In another embodiment of this invention, the insert may have an average diameter or thickness from about 10% to about 90% of the diameter or thickness of the core. In yet another embodiment of this invention, the core may comprise a plurality of inserts.

In another embodiment, the insert may have an average diameter, length, or thickness greater than about 90% of the diameter or thickness of the core, for example the insert may have an average length greater than about 100% of the thickness of the core.

In another embodiment of the invention, the core, the insert (if employed), the inlaid portion or any combination thereof may comprise a microelectronic device (e.g. an electronic “chip”) which may be used as an active component or to control, for example, the rate of release of active ingredients within the core or insert in response to an input signal. Examples of such microelectronic devices are as follows:

(1) Integrated, self-regulating responsive therapeutic devices including biosensors, electronic feedback and drug/countermeasure release devices which are fully integrated. Such devices eliminate the need for telemetry and human intervention, and are disclosed, for example, at www.chiprx.com/products.html, which is incorporated herein by reference;

(2) Miniaturized diagnostic imaging systems which comprise a swallowable capsule containing a video camera, and are disclosed, for example, at www.givenimaging.com/usa/default.asp, which is incorporated herein by reference;

(3) Subcutaneous glucose monitors which comprise implantable or insertable sensor devices which detect changes in glucose concentration within intestinal fluid, and communicate to an external detector and data storage device. Such devices are disclosed, for example, at www.applied-medical.co.uk/glucose.htm, which is incorporated herein by reference;

(4) Microdisplay vision aid devices encapsulated in an artificial intraocular lens. Such devices include a receiver for power supply, data and clock recovery, and a miniature LED array flip-chip bonded to a silicon CMOS driver circuit and micro optics, and are disclosed, for example, at http://ios.oe.uni-duisberg.de/e/, which is incorporated herein by reference. The microdisplay device receives a bit-stream+energy wireless signal from a high dynamic range CMOS camera placed outside the eye which generates a digital black & white picture which is converted by a digital signal processing unit (DAP) into a serial bit-stream with a data rate of approximately 1 Mbit/s. The image is projected onto the retina;

(5) Microchips used to stimulate damaged retinal cells, allowing them to send visual signals to the brain for patients with macular degeneration or other retinal disorders. The chip is 2 mm×25 microns, and contains approximately 5,000 microscopic solar cells (“microphotodiodes”), each with its own stimulating electrode. These microphotodiodes convert the light energy from images into electrical chemical impulses that stimulate the remaining functional cells of the retina in patients with AMD and RP. Such microchips are disclosed, for example, at www.optobionics.com/artificialretina.htm, which is incorporated herein by reference;

(6) Disposable “smart needles” for breast biopsies which display results in real time. The device fits into a 20 to 21 gauge disposable needle that is connected to a computer, as the needle is inserted into the suspicious lesion. The device measures oxygen partial pressure, electrical impedance, temperature, and light scattering and absorption properties including deoxygenated hemoglobin, vascularization, and tissue density. Because of the accuracy benefits from the six simultaneous measurements, and real-time nature of the device, it is expected to exceed the accuracy levels achieved by the core needle biopsy procedure and approach the high level of accuracy associated with surgical biopsies. Further, if cancer is found, the device can be configured to deliver various therapies such as cancer markers, laser heat, cryogenics, drugs, and radioactive seeds. Such devices are disclosed, for example, at www.bioluminate.com/description.html, which is incorporated herein by reference; and

(7) Personal UV-B recorders, which are instrument grade devices for measuring and recording UVB exposure and fit into a wrist-watch face. They may also be worn as a patch.

The core may be in a variety of different shapes and densities. In one embodiment, the core may have a density of about 0.7 g/cc to about 3.0 g/cc. With respect to different shapes, in one embodiment the core may be in the shape of a truncated cone. In other embodiments the core may be shaped as a polyhedron, such as a cube, pyramid, prism, or the like; or may have the geometry of a space figure with some non-flat faces, such as a cone, cylinder, sphere, torus, or the like. Exemplary core shapes which may be employed include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference) as follows (the tablet shape corresponds inversely to the shape of the compression tooling):
Shallow Concave.
Standard Concave.
Deep Concave.
Extra Deep Concave.
Modified Ball Concave.
Standard Concave Bisect.
Standard Concave Double Bisect.
Standard Concave European Bisect.
Standard Concave Partial Bisect.
Double Radius.
Bevel & Concave.
Flat Plain.
Flat-Faced-Beveled Edge (F.F.B.E.).
F.F.B.E. Bisect.
F.F.B.E. Double Bisect.
Ring.
Dimple.
Ellipse.
Oval.
Capsule.
Rectangle.
Square.
Triangle.
Hexagon.
Pentagon.
Octagon.
Diamond.
Arrowhead.
Bullet.
Barrel.
Half Moon.
Shield.
Heart.
Almond.
House/Home Plate.
Parallelogram.
Trapezoid.
Figure 8/Bar Bell.
Bow Tie.
Uneven Triangle.

The core or sub-core may optionally be at least partially covered by a compressed, molded, or sprayed sub-coating. However, in another embodiment, the core may be substantially free of the subcoating, i.e., there is no subcoating located between the outer surface of the core and the inner surface of the shell. Any composition suitable for film-coating a tablet may be used as a subcoating according to the present invention. Examples of suitable subcoatings include, but are not limited to, those disclosed in, for example, U.S. Pat. Nos. 4,683,256, 4,543,370, 4,643,894, 4,828,841, 4,725,441, 4,802,924, 5,630,871, and 6,274,162. Additional suitable subcoatings may include one or more of the following ingredients: cellulose ethers such as hydroxypropylmethylcellulose, hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as polysorbate-80, sodium lauryl sulfate and dioctyl-sodium sulfosuccinate; polycarbohydrates, pigments, and opacifiers.

In one embodiment, the subcoating may be comprised of, based upon the total weight of subcoating, from about 2 percent to about 8 percent, e.g. from about 4 percent to about 6 percent, of a water-soluble cellulose ether; and from about 0.1 percent to about 1 percent of castor oil, as disclosed in U.S. Pat. No. 5,658,589. In another embodiment, the subcoating may be comprised of, based upon the total weight of the subcoating, from about 20 percent to about 50 percent, e.g., from about 25 percent to about 40 percent of HPMC; from about 45 percent to about 75 percent, e.g., from about 50 percent to about 70 percent of maltodextrin; and from about 1 percent to about 10 percent, e.g., from about 5 percent to about 10 percent of PEG 400.

In one embodiment, the subcoating and/or the top coating may comprise an effect pigment that acts to maximize the reflectance of the core. Examples of suitable effect pigments include, but are not limited to, platy titanium dioxide, such as that disclosed in U.S. Pat. No. 6,627,212; and transition metal oxide coated platy mica such as that commercially available from EMD Chemicals Inc. under the tradename, “CANDURIN.” See also Pfaff, G. and Reynders, P., “Angle-dependent Optical Effects Deriving from Submicron Structures of Films and Pigments,” 99 Chem. Rev. 1963-1981 (1999). In embodiments wherein the dosage form contains a subcoating, the dosage form may contain, based upon the total weight of the dosage form, from about 1 percent to about 5 percent of the subcoating.

In one embodiment of the invention, only the core comprises one or more active ingredients. In another embodiment of this invention, only the second portion of the shell comprises one or more active ingredients. In yet another embodiment of this invention, only the insert comprises one or more active ingredients. In yet another embodiment of this invention, both the core and the first shell portion and/or the second shell portion comprise one or more active ingredients. In yet another embodiment of this invention, one or more of the core, the first shell portion, the second shell portion, or the insert comprises one or more of the active ingredients. Optionally, any of the coatings may further comprise one or more active ingredients. Core 4 may optionally also contain an active ingredient, which may be the same or different than the active ingredient contained in first shell portion 14 and second shell portion 15.

The first and second portions of the coating may be made from the aforementioned thermally responsive materials, which for food and pharmaceutical uses may be any material that has been approved for use in foods and pharmaceuticals and can be molded, including for example, film formers, low-melting hydrophobic materials, gelling polymers, thickeners, plasticizers, adjuvants, and excipients.

The flowable material suitable for use in the first portion 14 and the second portions 15 must be able to retain the randomized pattern when exposed to humidity and temperature variations typically encountered during storage, shipment, and use of the dosage forms worldwide. In addition, these flowable materials should easily and cleanly release from the mold after the coated dosage form has cooled and set.

In one embodiment, either the first portion 14 or the second portion 15 may optionally comprise a flavoring agent or sensate. As used herein, a “sensate” is a chemical agent that elicits a sensory effect in the mouth, nose, and/or throat other than aroma or flavor. Examples of such sensory effects include, but are not limited to, cooling, warming, tingling, mouth watering (succulent), astringent, and the like. Sensate agents suitable for use in the present invention are commercially available and may be purchased from, for example, International Flavor & Fragrances.

In one embodiment, at least one of the first or second portions comprises at least about 50%, e.g. at least about 80%, or at least about 90% of a material selected from film formers, gelling polymers, low-melting hydrophobic materials, non-crystallizable sugars or sugar alcohols, and mixtures thereof. In another embodiment, at least one of the first or second portions comprises at least about 50%, e.g. at least about 80% or at least about 90% of a material selected from film formers, gelling polymers, low-melting hydrophobic materials, and mixtures thereof.

In one embodiment of the invention, the flowable material comprises gelatin as a gelling polymer. Gelatin is a natural, thermogelling polymer. Two types of gelatin—Type A and Type B—are commonly used. Type A gelatin is a derivative of acid-treated raw materials. Type B gelatin is a derivative of alkali-treated raw materials. The moisture content of gelatin, as well as its Bloom strength, composition and original gelatin processing conditions, determine its transition temperature between liquid and solid. Bloom is a standard measure of the strength of a gelatin gel, and is roughly correlated with molecular weight. Bloom is defined as the weight in grams required to move a half-inch diameter plastic plunger 4 mm into a 6.67% gelatin gel that has been held at 10° C. for 17 hours. In one embodiment, the flowable material is an aqueous solution comprising 20% 275 Bloom pork skin gelatin, 20% 250 Bloom Bone Gelatin, and approximately 60% water. In one embodiment, at least one of the first portions or second portions comprises gelatin having a Bloom of about 150 to about 300, e.g., from about 200 to about 275.

In another embodiment of the invention, at least one of the first portions or second portions of the dosage form comprises at least about 80%, e.g. at least about 90%, of a material selected from film formers, gelling polymers (hydrocolloids), thermoplastic materials, low-melting hydrophobic materials, non-crystallizable sugars, and mixtures thereof.

In one embodiment, the first portion 14 and second portion 15 are applied to the core at substantially the same time. Alternatively, the first portion and second portion may be applied sequentially to the outer core surface 3.

In one embodiment wherein the randomization pattern of the shell is demonstrated by having a first color in the first portion 14 and second color in the second portion 15, the colors may be added to the aforementioned flowable materials in the form of lakes or dyes, depending upon the shade of color desired. In another embodiment, the color in at least one of the first and/or second portions may be present in the form of a fluorescent die. In another embodiment wherein anti-counterfeit measures are of concern, the solution that forms the first and/or second portion may further include an agent that could be visualized under wavelengths other than those of white light, e.g. black light or infrared.

In another embodiment, the randomization pattern of the shell is demonstrated by having a 10 percent or greater level of opacity in the second portion relative to the opacity of the first portion. The amount of opacity can be adjusted to the level desired through the addition of an opacifier, such as titanium dioxide.

The shell containing a randomized pattern may be applied to the surface of the core via any molding method known in the art. In one embodiment of the invention, the coating is applied to the surface of the dosage form using thermal setting injection molding or thermal cycle injection molding as described above and in, for example, US Patent Publication No. 2003/0068367. In an alternate embodiment, the coating may be applied to the core via known dipping methods as disclosed in, for example, U.S. Pat. No. 4,820,524. In yet another alternate embodiment, a film having a randomized pattern may be enrobed onto a core using known enrobing methods as disclosed in, for example, U.S. Pat. No. 6,482,516 and U.S. Pat. No. 5,146,730.

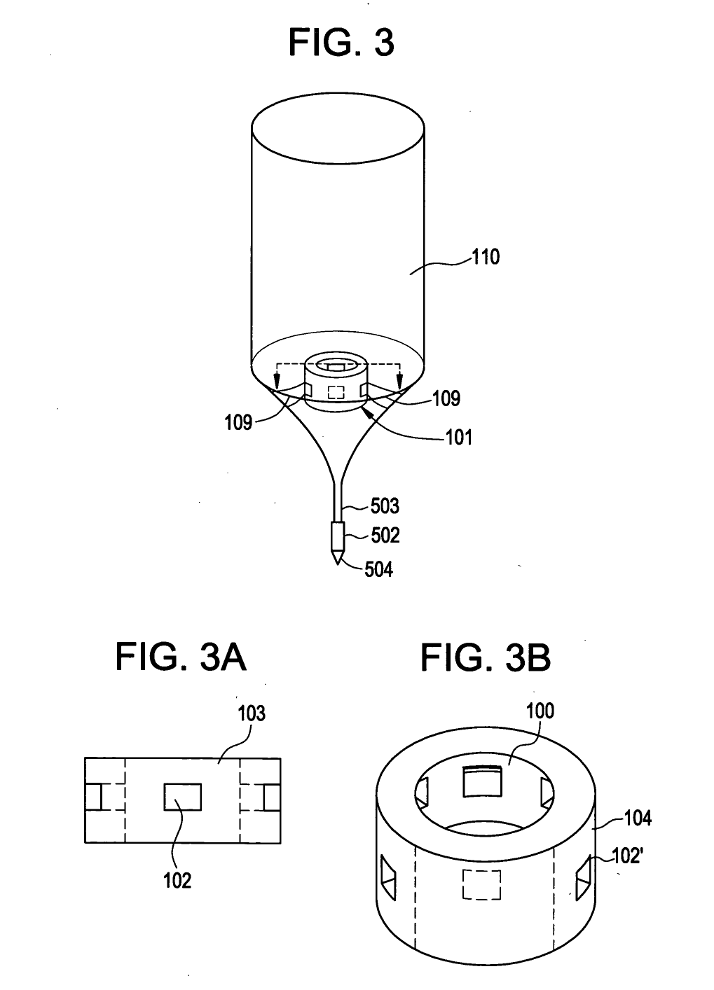

As illustrated in FIGS. 3-5, the flowable material may be kept in one or more feeding tanks or reservoirs 110 until the desired time for coating the core 4 of the dosage form 2. In one embodiment as shown in FIG. 3, the flowable material may be transported from the reservoir 110 to the desired location on the dosage form via one or more feedlines 503 connected to one or more injector ports 502. The reservoir 110, which contains a first shell solution having a first feature, is fitted with a diffusion device 101 containing a second shell solution having a second feature. In one embodiment, wherein mixing of the solutions at the point of application to the substrate is of concern, the second shell portion solution may have a viscosity which is at least 10 percent greater than the viscosity of the first shell portion solution.

In one embodiment, the diffusion device 101 may be of any shape that has a central hole 100 therethrough. The shape of the central hole 100 is not critical, but the overall shape of the diffusion device 101 may be, for example, doughnut-like, square frame-like, rectangular frame-like, triangle frame-like, etc. In this embodiment, the device 101 has an internal device wall 103 with at least one passage hole 102 therethrough and/or at least one passage hole 102′ through its outside device wall 104. In an alternative embodiment (not shown), the diffusion device 101 may not have an internal device wall, but have at least one passage hole 102′ through its outside device wall 104. One skilled in the art would readily appreciate without undue experimentation that the number, size, and arrangement of the holes 102 will affect the amount and shape of the second portion in the resulting coating 6. The diffusion device 101 may be comprised of any pharmaceutically-acceptable material that does not interact with the solutions such as, for example, metals such as stainless steel, aluminum, steel, and titanium; non-metal materials such as plastics, rubber, and polymers; or any of the above coated with a non-stick surface material.

In one embodiment, a block comprised of a water dispersable thermally responsive material in a solid or semi-solid state may be inserted into the diffusion device 101. As used herein, “solid or semi-solid state” shall mean a hard or soft state that is incapable of flowing in a manner similar to a liquid or gas. In this embodiment, the block may be formed of a material which will form the second shell portion and which is capable of slowly dissolving into the first shell portion solution when placed inside of the diffusion device 101 as illustrated in FIG. 3 and exposed to the positive pressure of the first solution. Materials suitable for the block in this embodiment include but are not limited to, for example, colorants combined with any of the thermally responsive materials such as gelatin, carrageenan, hypromellose, gellan gum, meltable waxes, and polyethylene glycol. In one embodiment, the block may be prepared by forming an aqueous solution of gelatin and up to at least about 10 weight percent colorant at a temperature above 35° C., then gelling the resulting solution at a temperature between 10° C. and 30° C. in a rubber, plastic or metal mold prepared in the shape of the diffusion device. The gelled block may then be dried between 20° C. and 50° C., and removed from the mold.

The diffusion device 101 may be affixed to any location within the reservoir 110 that will enable its contents to slowly flow into the surrounding solution. In one embodiment, the diffusion device 101 is secured proximate to the bottom of the feeding tank 110 via any securing means known in the art such as, for example, stainless steel brackets 109 attaching the internal side walls of the feeding tank to the exterior wall of the diffusion device.

During operation, the temperature of first shell solution may range between about 30° C. to about 200° C., e.g. about 50° C. to about 90° C., and the temperature of the second shell solution may range between about 20° C. to about 200° C., e.g. about 20° C. to about 90° C. As the first solution passes through the central hole 100 of the device 101, it contacts the second solution at the passage holes 102, and causes the second solution to melt and blend into the first coating solution. The resulting blended solution may be dispensed from the reservoir 110 through feedlines 503 connected to an injector port 502 having a valve 504, then applied to a substrate via injection molding as disclosed in, for example, United States Patent Publication No. 2003/0068367.

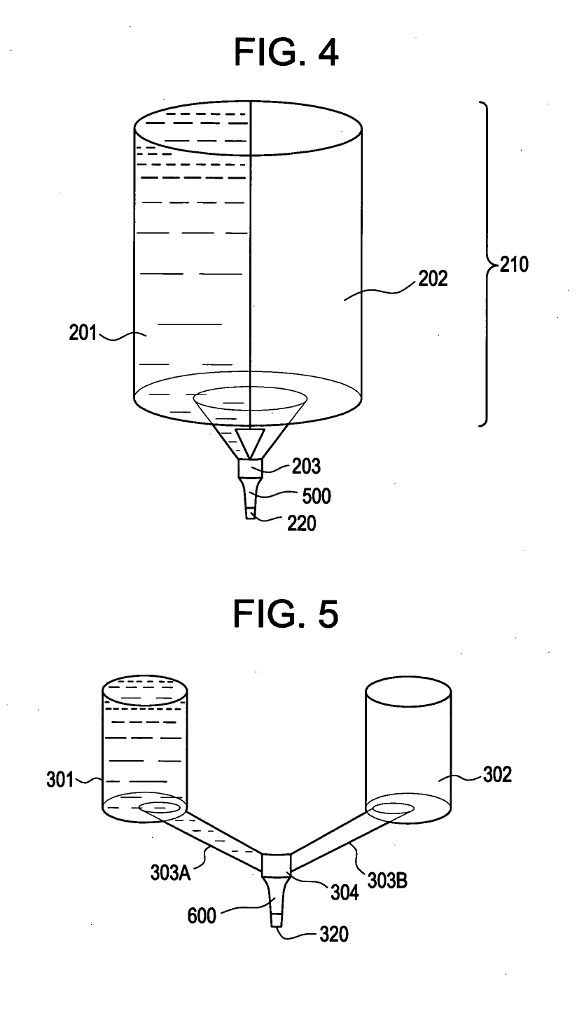

FIG. 4 illustrates another embodiment wherein one reservoir 210 having a first compartment 201 and a second compartment 202 may be charged with a first shell solution having a first feature and a second shell solution having a second feature, respectively. Each of the solutions independently flows into a static mixer 203, which causes the second solution to blend into the first solution. The degree of mixing between the two solutions is controlled via the speed of the static mixer, which is thereby dependent on the flow rate of the two solutions into the mixer. One skilled in the art would readily appreciate that the flow rate of each solution may be controlled by the addition of positive air pressure into its reservoir 210, respectively, and typically the pressure may range from about 5 psi to about 50 psi. The resulting blended solution may be dispensed from the mixer 203 to an injector port 500 having a check valve 220, and applied to a substrate via injection molding.

In yet another alternative embodiment shown in FIG. 5, a first reservoir 301 containing a first shell solution and a second reservoir 302 containing a second shell solution are connected via feedlines 303A, 303B, equipped with a static mixer 304 The degree of mixing between the two solutions is controlled via the speed of the static mixer, which is thereby dependent on the flow rate of the two solutions into the mixer. One skilled in the art would readily appreciate that the flow rate of each solution may be controlled by the addition of positive air pressure into its reservoir 210, respectively, and typically the pressure may range from about 5 psi to about 50 psi. The resulting blended solution may be dispensed from the mixer 304 to an injector port 600 having a check valve 320, then applied to a substrate via injection molding.

In another embodiment, the blended solution resulting from any of the processes described above may be added to a separate reservoir (not shown) for application to the substrate via a dipping process as disclosed in, for example, U.S. Pat. No. 4,820,524. In another alternative embodiment, the resulting blended solution may be dried into a pre-made film, then applied to a substrate in an enrobing process as disclosed in, for example, U.S. Pat. No. 6,482,516 and U.S. Pat. No. 5,146,730.

Although not shown, it may be possible to arrange a multitude of reservoirs and feedlines for use in embodiments requiring more than two different solutions.

As shown in greater detail in FIGS. 48-52 of United States Patent Publication No. 2003/0068367, a tip or valve 504 located at the bottom of each injector port 502 passes through a hole 505 in the surface of the upper mold 506. According to the present invention, the desired amount of partially blended flowable material may pass through the tip or valve 504 and into the cavity 501. The valve 504 may then be closed, which thus closes the hole 505 during the molding period. The location of the hole 505 is not critical, so long as it permits the flowable material to be injected into mold containing the dosage form 510. See also FIGS. 52, 53, and 54 of W003/028990.

The upper mold 506 is engaged with either a holder or “collet” for the dosage form or a lower mold 507. Although the upper mold 506 and the lower mold 507 are illustrated as moving in a longitudinal manner in order to produce the molded dosage form, the operational direction of these pieces is not critical.

After the mold is filled with the desired amount of flowable material, the closed mold may then be adjusted to an appropriate temperature and for a time sufficient to set the flowable material on the dosage form. Although these parameters may vary depending upon, for example, the type and amount of flowable material, typically the molding temperature is from about 50° C. to about 120° C. and the molding time is from about 1 seconds to about 10 seconds.

In one embodiment, the dosage form contains a core having two faces and a belly band therebetween, and a shell having a thickness from about 100 microns to about 400 microns that substantially covers at least one face surface. The other face surface may be compositionally and/or visually different from the shell. The shell may contain, based upon the total weight of said shell, less than about 50 percent crystallizable sugar.

As illustrated in FIG. 2, after the coating having a randomized pattern is applied to the desired location(s) on the core, an optional top coating 13 may then be applied to the outer core surface 3 via any of the above-described coating application methods such as, for example, spraying, molding, or dipping, and at a temperature below the melting temperature of the core material. In embodiments wherein the core is a compressed powder blend, such temperature may typically range from about 5° C. to about 120° C.

As illustrated in FIG. 2, the dosage form may contain an optional clear or semi-transparent top coating 16 that resides on the upper first surface 8 of the shell 6. Suitable polymers for inclusion in top coatings include polyvinylalcohol (PVA); water soluble polycarbohydrates such as hydroxypropyl starch, hydroxyethyl starch, pullulan, methylethyl starch, carboxymethyl starch, pre-gelatinized starches, and film-forming modified starches; water swellable cellulose derivatives such as hydroxypropyl cellulose (HPC), hydroxypropylmethyl cellulose (HPMC), methyl cellulose (MC), hydroxyethylmethylcellulose (HEMC), hydroxybutylmethylcellulose (HBMC), hydroxyethylethylcellulose (HEEC), and hydroxyethylhydroxypropylmethyl cellulose (HEMPMC); water soluble copolymers such as methacrylic acid and methacrylate ester copolymers, polyvinyl alcohol and polyethylene glycol copolymers, polyethylene oxide and polyvinylpyrrolidone copolymers; polyvinylpyrrolidone and polyvinylacetate copolymers; and derivatives and combinations thereof. Suitable film-forming water insoluble polymers for inclusion in top coatings include for example ethylcellulose, polyvinyl alcohols, polyvinyl acetate, polycaprolactones, cellulose acetate and its derivatives, acrylates, methacrylates, acrylic acid copolymers; and the like and derivatives, copolymers, and combinations thereof. Suitable film-forming pH-dependent polymers for inclusion in top-coatings include enteric cellulose derivatives, such as for example hydroxypropyl methylcellulose phthalate, hydroxypropyl methylcellulose acetate succinate, cellulose acetate phthalate; natural resins, such as shellac and zein; enteric acetate derivatives such as for example polyvinylacetate phthalate, cellulose acetate phthalate, acetaldehyde dimethylcellulose acetate; and enteric acrylate derivatives such as for example polymethacrylate-based polymers such as poly(methacrylic acid, methyl methacrylate) 1:2, which is commercially available from Rohm Pharma GmbH under the tradename, “EUDRAGIT S;” and poly(methacrylic acid, methyl methacrylate) 1:1, which is commercially available from Rohm Pharma GmbH under the tradename, EUDRAGIT L; poly (butyl methacrylate (dimethylaminoethyl)methacrylate, methyl methacrylate), which is commercially available from Rohm Pharma GmbH under the tradename, “EUDRAGIT E;” and the like, and derivatives, salts, copolymers, and combinations thereof.

In one embodiment, top coating 13 includes those coatings having a high rigidity, i.e., e.g., those coatings having a yield value sufficient to prevent deformation of the randomized pattern when exposed to normal manufacturing, handling, shipping, storage, and usage conditions. Suitable top coatings having high rigidity include film formers, such as for example, the high tensile strength film-formers well known in the art. Examples of suitable high tensile strength film-formers include, but are not limited to methacrylic acid and methacrylate ester copolymers; polyvinylpyrrolidone; cellulose acetate; hydroxypropylmethylcellulose (“HPMC”), polyethylene oxide and polyvinylalcohol, which is commercially available from BASF under the tradename, “Kollicoat IR”; ethylcellulose; polyvinyl alcohols; and copolymers and mixtures thereof.

In one embodiment, the top coatings may include the water-soluble high rigidity film formers selected from HPMC, polyvinylpyrrolidone, the aminoalkyl-methacrylate copolymers marketed under the trade mark, “EUDRAGIT E,” and copolymers and mixtures thereof.

In embodiments wherein high clarity is of particular concern, the top coatings may include the high clarity high-rigidity film formers selected from the acrylates such as the aminoalkyl-methacrylate copolymers marketed under the trademark, “EUDRAGIT E,” polyvinylpyrrolidone, cellulose acetate, polyethylene oxide and polyvinylalcohol, ethylcellulose, and polyvinyl alcohol shellac.

In general, the thickness of the top coating may range from about 50 microns to about 200 microns, and the rigidity of the top coating will increase as the thickness is increased.

In one embodiment, the dosage form may contain, based upon the total dry weight of the dosage form, from about 1 percent to about 40 percent, e.g. from about 5 to about 30 percent of the shell having a randomized pattern, and from about 0.1 percent to about 10, e.g. from about 2 percent to about 10 percent of the top coating. The shell may contain, based upon the total weight of the shell, from about 50 percent to about 99 percent, e.g. from about 50 percent to about 80 percent of the first portion, and from about 1 percent to about 50 percent, e.g. from about 1 percent to about 40 percent, of the second portion.

The top coating 13 may be applied via any means known in the art such as, for example, spray coating as disclosed in, U.S. Pat. Nos. 4,683,256, 4,543,370, 4,643,894, 4,828,841, 4,725,441, 4,802,924, 5,630,871, and 6,274,162; dip coating as disclosed in, U.S. Pat. Nos. 5,089,270; 5,213,738; 4,820,524; 4,867,983; and 4,966,771; or injection molding as disclosed in, US application 2003-0219484 A1.

Advantageously, the dosage forms produced in accordance with the embodiments of the present invention may possess a unique, product identifying appearance, which not only help the user to identify the brand but also help to control and detect counterfeit dosage forms.

Further, the dosage forms may also advantageously provide unique visual and color effects and images to dosage forms, as well as to other toiletry, cosmetic, healthcare, and foodstuffs, such that they possess a unique appearance without the use of inedible metal, dye, color, and ink pigments. In one embodiment, the brightness of the shell on a dosage form may further be enhanced by using a core having a shiny light colored, e.g. white, reflective surface. As used herein, “shiny” or “highly glossy” means that the core, substrate, or dosage form possesses a surface gloss of at least 200, for example between about 200 to about 300. “Surface gloss,” as used herein, refers to the amount of light reflectance as measured at a 60 degree incident angle using the method set forth in Example 7 of United States Patent Application Publication No. 20030072731. For example, in embodiments wherein a highly glossy effect is desired, the core may be comprised of a polyol such as sorbitol, xylitol, mannitol, and the like, or may be coated with a subcoating comprised of, for example, pullulan and other subcoatings as disclosed in U.S. Pat. Nos. 6,248,391; 6,274,162; 5,468,561; 6,448,323; 6,183,808; and 5,662,732; and WO 2004 073582.

In addition, the dosage forms of the present invention beneficially may be made with apparatus and processes that are not only economical to use, but also are compatible with current production techniques.

This invention will be further illustrated by the following examples, which are not meant to limit the invention in any way. Although this invention has been illustrated by reference to specific embodiments, it will be apparent to those skilled in the art that various changes and modifications may be made which clearly fall within the scope of this invention.

### EXAMPLES

### Example 1

### Compressed Acetaminophen Tablets with Randomized Coating

Acetaminophen tablets having the formula set forth in Table A below are compressed on a rotary tablet press.

TABLE A
Tablet Core Formulation

| Ingredient | mg/tablet core |
|---|---|
| Paracetamol DC273N (P.G.S.)- US* | 529.1 |
| Sodium Starch Glycolate NF-Explotab | 25.0 |
| Magnesium Stearate NF | 2.0 |
| TOTAL CORE | 556.1 |

*granulation available from Mallinckrodt

Once formed, the tablet is transferred to an injection molding apparatus, where the tablet is placed in a mold cavity such that the portion of the tablet is located under an injector tip.

Preparation of White Gelatin Solution

Pork skin gelatin granules (275 bloom), which are commercially available from Geltia Corporation, are dissolved in a vessel of warm water at a temperature of 60° C. with mixing at a speed of about 50 RPM to yield a 35% solution. A white colorant, which is commercially available from Colorcon, Inc. under the tradename, “Opatint White,” is added thereto to yield a solution containing 1% colorant.

The resulting white solution, which has a viscosity of about 2500 cps to about 3000 cps, is maintained at 55° C. prior to injection molding.

Preparation of Blue Gelatin Solution

Pork skin gelatin granules (275 bloom), which are commercially available from Gelita Corporation, are dissolved in a vessel of warm water at a temperature of 60° C. with mixing at a speed of about 200 RPM to yield a 35% solution. A blue colorant, which is commercially available from Colorcon, Inc. under the tradename, “Opatint Blue,” is added thereto to yield a solution containing 1.5% colorant.

The resulting blue solution, which has a viscosity of about 2500 cps to about 3000 cps, is maintained at 55° C. prior to injection molding.

Preparation of Blue Gelatin Block

About 800 g portion of the blue gelatin solution is then added to a pre-formed, cylindrical rubber mold having a 50 mm central hole, and gelled at a temperature of 20° C. The gelled mass is then removed and dried at 25° C. for 24 hours to form a gelatin block having a dry weight of about 300 g.

Injection Molding Process

The blue gelatin block is inserted into a cylindrical stainless steel diffusion device having a bottom and three, 5 mm holes along its internal cylindrical wall. A lid is then placed on the device, and the device is inserted into a feed tank as illustrated in FIG. 3. The blue gelatin block is at a temperature of about 25° C. Two liters of the white gelatin solution are then charged into the feed tank, which is maintained at a temperature of 55° C. As the valve 504 is opened, the air pressure on the feed tank is increased to about 30 psi. The white gelatin solution then slowly passes through the central hole 100 of the device 101 and contacts the blue gel along the surface of the internal device wall 103. As the blue gelatin is melted by the proximately flowing white gelatin solution, it diffuses out of the passage holes 102 and begins to blend and diffuse into the white gelatin solution. The white solution, in conjunction with diffused blue gel, flows to the injector port 502, which is connected to a core-containing mold as illustrated in FIGS. 48-52 of United States Patent Publication No 2003/0068367. After approximately 132 milliliters of blended solution at a temperature of about 10° C. is injected into the mold, which is also maintained at a temperature of about 10° C., the mold is removed. The resulting coated core having a randomized pattern appearance is then dried under conditions of 22° C. and 35% RH. The coating weight gained is, based upon the original weight of the uncoated compressed tablet, from about 2 percent to about 40 percent.

### Example 2

### Compressed Acetaminophen Tablets with Randomized Coating

The process of Example 1 is repeated, but with the substitution of a two-compartment feed tank as illustrated in FIG. 4 for the single feed tank and diffusion device 101 unit described in Example 1. One compartment of the two-compartment feed tank is charged with the white gelatin solution of Example 1, and the other compartment is charged with the blue gelatin solution of Example 1. The white gelatin solution and blue gelatin solution flow into a static mixer 203, which partially mixes the two solutions in-line. The flow rate of the two solutions into the static mixer 203 is about 2100 milliliters per 20 seconds. The resulting mixed solution then flows to the injector port 500.

### Example 3

### Compressed Acetaminophen Tablets with Randomized Coating

The process of Example 1 is repeated, but with the substitution of a two, independent feed tanks as illustrated in FIG. 5 for the single feedtank and device 101 unit described in Example 1. One of the feed tanks is charged with the white gelatin solution of Example 1, and the other feed tank is charged with the blue gelatin solution of Example 1. The white gelatin solution and blue gelatin solution flow through transport tubing 303 equipped with a static mixer 304, which partially combines the two solutions. The flow rate of the two solutions into the static mixer 304 is about 2100 milliliters per 20 seconds. The resulting mixed solution then flows to the injector port 500.

### Example 4

### Manufacturing Process of Tablets with Randomized Coating

The process of Example 1 is repeated, but with using a continuous injection molding manufacturing process, which includes an apparatus having two thermal cycle molding modules linked in series via a transfer device as described at pages 14-16 of United States Patent Publication No. 2003/0068367 (“'367 Publication”).

The thermal cycle molding modules have the general configuration shown in FIG. 3 and pages 27-51 of United States Patent Publication No. 2003/0086973 (“'973 Publication”), which depicts a thermal cycle molding module comprising a rotor around which a plurality of mold units are disposed. The thermal cycle molding modules include two separate reservoirs (see FIG. 4 of '973 Publication) for holding the white gelatin and blue gelatin materials. In addition, each thermal cycle molding module is provided with a temperature control system for rapidly heating and cooling the mold units as illustrated in FIGS. 55 and 56 of the '973 Publication.

The transfer device, which is illustrated in FIG. 3 and described on pages 51-57 of WO 03/28989, comprises a plurality of transfer units attached in cantilever fashion to a belt. See Id., FIGS. 68 and 69. The transfer device rotates and operates in sync with the thermal cycle molding modules to which it is coupled. Transfer units comprise retainers for holding the cores as they travel around the transfer device.

The cores of Example 1 are transferred via this transfer device to the second molding module, which applies the two shell portions to the core in a randomized pattern. The second thermal cycle molding module is of the type shown in FIG. 28A of the '973 Publication. The mold units of the second thermal cycle molding module comprise upper mold assemblies, rotatable center mold assemblies and lower mold assemblies as shown in FIG. 28C of the '973 Publication. The cores of Example 1 are continuously transferred to the mold assemblies, which then close over the cores.

At the beginning of the molding cycle (rotor at the 0 degree position) the mold assemblies are in the open position. The center mold assembly receives the compressed core from a compression module that was transferred via a transfer device. As the rotor continues to revolve, the upper mold assembly closes against center mold assembly. The partially blended blue and white gelatin solution, which is at a temperature of about 55° C., is then injected into the mold cavity created by union of the mold assemblies, which is maintained at a temperature of about 10° C. After the solutions are cooled, the mold assemblies open with the partially coated dosage forms remaining in the upper mold assembly 4. Upon further revolution of the rotor, the center mold assembly rotates 180 degrees. As the rotor moves past 180 degrees, the mold assemblies again close, and the uncoated portion of the compressed dosage form is covered with the blended blue and white gelatin solution to form the second half of the shell. After the shell sets or hardens on the second half of the compressed dosage form, the mold assemblies again open, and the coated compressed dosage form is ejected from the molding module.

We claim:

1. A pharmaceutical dosage form comprised of:
a) a core having an upper face and a lower face, said upper face having an outer upper face surface and said lower face having an outer lower face surface; and
b) a shell substantially covering at least one of said faces, said shell having a first portion and a second portion;
wherein the shell is comprised of based upon the total weight of the shell, from at least about 50 percent of a thermally responsive material, and said first and second portions of said shell being visually distinct from each other and arranged in a randomized pattern.

2. The dosage form of claim 1, wherein said first portion has a first visual appearance characterized by a first color and said second portion has a second visual appearance characterized a by a second color that is different from said first color.

3. The dosage form of claim 1, wherein the core is further comprised of a bellyband between the upper face and the lower face, and the thickness of the shell at the bellyband is about 50% less than the thickness of the shell at the face.

4. The dosage form of claim 1, wherein the shell possesses a thickness of about 10 microns to about 1000 microns.

5. The dosage form of claim 1, wherein the shell possesses a melting point of greater than about 50° C.

6. The dosage form of claim 1, wherein the randomized pattern is marbled.

7. The dosage form of claim 1, wherein the randomized pattern is swirled.

8. The dosage form of claim 1, wherein the first portion is comprised of gelatin and a colorant.

9. The dosage form of claim 1, wherein the first portion is comprised of polyethylene glycol and a colorant.

10. The pharmaceutical dosage form of claim 1, wherein the core has a density of about 0.7 g/cc to about 3.0 g/cc.

11. The pharmaceutical dosage form of claim 1, wherein the shell has a moisture uptake value of less than about 0.65% when exposed to conditions of 40° C. and 75% relative humidity for 60 minutes.

12. The dosage form of claim 1, wherein said first portion has a first visual appearance characterized by a first color and said second portion has a second visual appearance characterized a by a second, pearlescent color.

13. The dosage form of claim 1, wherein the shell is comprised of a low temperature, thermally responsive material.

14. The dosage form of claim 1, wherein the first portion has an opacity that is at least 10% greater than the opacity of the second portion.

15. A pharmaceutical dosage form comprised of, based upon the total weight of the dosage form:
a) from about 60 percent to about 99 percent of a core having an upper face and a lower face, said upper face having an outer upper face surface and said lower face having an outer lower face surface; and
b) from about 1 percent to about 40 percent of a shell substantially covering at least one of said faces, said shell comprised of, based upon the total weight of the shell, from about 50 to about 99 percent of a first portion and about 1 to 50 percent of a second portion;
wherein the core is comprised of an active ingredient selected from acetaminophen, ibuprofen, loperamide, simethicone, pseudoephedrine, famotidine, phenylephrine, chlorpheniramine, dextromethorphan, diphenhydramine, guaifenesin, calcium carbonate, menthol, aspirin, and mixtures thereof, and the shell is comprised of, based upon the total weight of the shell, from at least about 50 percent of a low temperature, thermally responsive material, and said first and second portions of said shell being visually distinct from each other and arranged in a randomized pattern.

16. A process for producing a shell having a randomized pattern on a pharmaceutical dosage form comprising:
a) flowing a first solution comprised of a first low temperature thermally responsive material through an opening in a metering device;
b) flowing a second solution comprised of a second low temperature thermally responsive material through the opening;
c) forming a shell having a randomized pattern on a pharmaceutical core located proximate to the opening, said shell comprised of a first portion formed from the first solution and a second portion formed from the second solution,
wherein the first portion and the second portion are visually distinct from each other.

17. The process of claim 16 wherein step a and step b occur at substantially the same time.
